(19) United States
(12) Patent Application Publication
(10) Pub. No.: US 2026/0259162 A1
(43) Pub. Date: Sep. 3, 2026

(54) A SINGLE CHIP SENSING SYSTEM TO DETECT MULTIPLE COMPONENTS IN AN AROMA

(71) Applicant: STRATUSCENT INC., Saint-Laurent (CA)

(72) Inventors: Ashok Prabhu Masilamani, Montreal (CA); Mojtaba Khomami Abadi, Montreal (CA); Fatemeh Yazdanpanah, Montreal (CA)

(73) Assignee: STRATUSCENT INC., Saint-Laurent, QC (CA)

(21) Appl. No.: 18/994,530

(22) PCT Filed: Jul. 19, 2023
(86) PCT No.: PCT/IB2023/000441

§ 371 Date: Jan. 14, 2025

Related U.S. Application Data
(60) Provisional application No. 63/391,183, filed on Jul. 21, 2022.

(51) Int. Cl. G01N 27/12

(52) U.S. Cl. CPC G01N 27/12

(57) ABSTRACT

The application discloses a chemical sensing system that comprises a plurality of sensors, present on a chip. The system further comprises a processor programmed to execute a plurality of artificial intelligence-based models. The sensors of the sensing system undergo at least one or physical or chemical change when the sensor is exposed to an aroma. The processor uses information about the observed physical and/or chemical changes of the sensors and a plurality of trained artificial intelligence models to recognize at least one component/chemical present in the aroma. The processor then uses the output of the trained models to detect the plurality of components of the aroma that the sensor is exposed to.

### FIELD OF INVENTION

The present application relates to a chemical sensing system used to detect a plurality of components of an aroma through a sensor chip and an artificial intelligence-based processor.

### BACKGROUND

An aroma is a coexisting combination of plurality of gases or chemicals, hereinafter referred to as components. The aroma may be generated by a chemical process, as a main product of the chemical process or as a by-product of the chemical process. In the environment, the emanation of just one gas from a chemical process is rare, due to the volatile and interactive nature of the chemicals, and hence the gases are usually present in the form of an aroma. The aroma may be characterised based on its attribute or human perception of the attribute or through the chemical composition of the aroma. Since aroma detection is important to determine the characteristic of a process that emanated the aroma, therefore a plurality of systems are being made to detect the aroma at perception level or at constituting chemicals level.

### SUMMARY

Some conventional chemical sensing systems detect specific chemicals present in an aroma. Such systems may come in the form of single gas sensors that comprise a sensor capable of detecting a specific gas or chemical. A limitation of these single gas sensors is the specificity of the sensor, with the sensor indicating the presence of a gas, even though the gas may not be present. This problem may arise because of the similar way in which a plurality of chemicals or gases interact with the detector.

Further, for some chemical sensing systems, the sensors of the systems may start to degrade as soon as the sensor is exposed to the gas or chemical, due to the nature of the interaction between the chemical or gas and the sensor. Due to this, these systems lose their sensitivity in a short period of time.

For a chemical sensing system that is capable of detecting multiple gases of an aroma, the system may be configured to detect only a few properties of the interaction of the chemicals/gases of aroma and the sensing system. This reduces the specificity of the chemical sensing system. Further, some types of sensors start to degrade during chemical interaction, therefore, the capability of detecting a plurality of chemicals is also lost over a short period of time.

Furthermore, if a plurality of sensors are used to make a chemical sensing system, wherein each of the sensors is specific to just one type of chemical, then the system becomes bulky. Also, the interaction between the aroma and a specific sensor may be hindered due to the position of the plurality of sensors in the chemical sensing system.

Some embodiments of the present disclosure solve one or more of the above problems by using a single chip-based sensing system wherein a plurality of sensors are present on one substrate. Further, the system described herein uses specially trained artificial intelligence (AI) models to infer the characteristics of the aroma from the interaction between the plurality of chemicals of the aroma and the plurality of sensors of the sensing system.

Some embodiments of the present disclosure describe a single chip sensing system that is used to detect multiple components in an aroma, wherein the aroma is in a gaseous or liquid form. The system comprises a plurality of sensors that are provided on a substrate of the chip. Each of the sensors of the sensing system is made from one or more composites that react with the aroma and undergo at least one of physical or chemical property changes. The change in physical and/or chemical properties is communicated with a processor of the sensing system, which translates the change in the physical and/or chemical properties of the plurality of sensors into the concentration of the plurality of components in the aroma, through a group of specially trained artificial intelligence models. The components may then be mapped to an aroma component map, such as an analyte map, a virus map, or a mould map, to detect the presence of moulds, viruses and other components/analytes present in the aroma. Based on the extent of the mapping, at least one of application-based control or notification may be issued or initiated.

In some embodiments, a single-chip sensing system for detecting a plurality of components of an aroma is provided. The system includes a sensor array including a plurality of sensors, wherein at least one sensor of the plurality of sensors is configured to react with at least one of the plurality of components of the aroma and generate a response, and a processor programmed to execute a plurality of artificial intelligence (AI) models trained to infer the plurality of components of the aroma from the response of the plurality of sensors of the sensor array. At least one model of the plurality of AI models is trained to detect at least one component of the aroma, and the at least one model is trained to detect the response of the sensors that are not associated with the component of the aroma. The plurality of AI models are trained to compare the response associated with a component and response that is not associated with the component and generate a confident inference about the presence of the component in the aroma.

In one instance, at least one model of the plurality of AI models comprises at least three sub-models that sequentially process at least a portion of the response from the plurality of sensors, to detect at least one component of the plurality of components of the aroma. In another instance, a first sub-model of the at least three sub-models is trained to generate a feature representation of at least a portion of the sensor response, a second sub-model of the at least three sub-models is trained to generate a latent representation of the feature representation of the at least a portion of the sensor response, and a third sub-model of the at least three sub-models is trained to create a straightened orthogonal representation of the latent representation. In another instance, at least one model of the plurality of AI models is trained to detect at least one of the plurality of components of the aroma from the straightened orthogonal representation by using a long short-term memory network-based model. In another instance, the plurality of sensors of the sensor array are chemo-resistive sensors, each of the chemo-resistive sensors comprising a composite including a polymeric composition and conducting nanoparticles, and wherein an amount of polymer in the composite and an amount conducting nanoparticles in the composite is provided to at least one of the plurality of AI models, to assess the response of the sensors, using the plurality of AI models, wherein at least one of the plurality of AI models is trained to associate a response of the sensor with a component of the aroma, through the composition of the composite of the sensor.

In some embodiments, a single chip sensing system configured to detect a plurality of components of a gaseous sample is provided. The system includes a sensor array comprising a plurality of sensors, wherein at least one sensor of the plurality of sensors is configured to react with at least one of the plurality of components of the gaseous sample and generate a response, and wherein the plurality of sensors of the sensor array are configured to react with each of the components of the plurality of components of the gaseous sample, and a processor programmed to execute a plurality of models trained to detect the plurality of components of the gaseous sample from the response of the plurality of sensors of the sensor array. A temporal sensor response created by a change in a collective response of the plurality of sensors of the sensor array is provided to the processor, and each of the models of the plurality of models is trained to associate a portion of the temporal sensor response with a component of the gaseous sample, thereby detecting the plurality of components of the gaseous sample.

In one instance, at least one model of the plurality of models of the processor comprises at least three sub-models, that sequentially process at least a portion of the response from the plurality of sensors, to detect at least one component of the plurality of components of the gaseous sample. In another instance, a first sub-model of the at least three sub-models is trained to generate a feature representation of at least a portion of the sensor response, a second sub-model of the at least three sub-models is trained to generate a latent representation of the feature representation of the at least a portion of the sensor response, and a third sub-model of the at least three sub-models is trained to create a straightened orthogonal representation of the latent representation. In another instance, at least one model is trained to detect at least one of the plurality of components of the gaseous sample from the straightened orthogonal representation by using a long short-term memory network-based model. In another instance, the plurality of sensors of the sensor array are chemo-resistive sensors, each of the chemo-resistive sensors comprising a composite including a polymeric composition and conducting nanoparticles, and wherein an amount of polymer in the composite and an amount conducting nanoparticles in the composite is provided to at least one of the plurality of models, to assess the response of the sensors, using the plurality of models, wherein at least one of the plurality of models is trained to associate a response of the sensor with a component of the gaseous sample, through the composition of the composite of the sensor.

### BRIEF DESCRIPTION OF THE DRAWINGS

FIG. 1 illustrates a process flowchart of the chemical sensing system, as per an embodiment of the application.

FIG. 2 illustrates aroma components information, generated by the chemical sensing system as per an embodiment of the application.

### DETAILED DESCRIPTION

Disclosed herewith is an aroma sensing system that may be used to detect the presence of a plurality of components in the aroma. The sensing system comprises a plurality of sensors that detect the presence of the components in the aroma. Each of the sensors of the plurality of sensors of the sensing system reacts with at least one of the plurality of components of the aroma and undergoes at least one of physical or chemical change. At least one of the physical and/or chemical change of the plurality of sensors is used to identify the components and the concentration of components in the aroma.

In some embodiments of the invention, the plurality of the sensors of the sensing system is present on a single substrate, wherein the sensor may be present in the form of a rectangular array. In some embodiments, the plurality of sensors may be present in multiple substrates, wherein each substrate may have a group of the plurality of sensors. The plurality of sensors of the sensor array may be sufficiently spaced apart from each other, thereby minimising the effect of change in at least one of physical or chemical change in one sensor to affect at least one of physical or chemical change of another sensor of the plurality of sensor of the sensor array.

In some embodiments of the invention, the plurality of sensors of the sensor array is positioned such that the position of the sensor array does not bias at least one of physical or chemical change of the sensors when the plurality of sensors reacts with the components of the aroma.

The sensing system may be used for sensing aromas that are in fluidic form (e.g., gaseous form). The sensors of the sensing system may be exposed to the aroma in an operational environment of the sensing system, wherein the components of the aroma react with the plurality of sensors of the sensing system.

In some embodiments of the invention, the sensing system is designed such that the level of exposure of each of the sensors to the aroma is the same. Further, each of the plurality of sensors of the sensing element may undergo at least one physical and chemical change during exposure.

In yet another embodiment, the sensors of the sensing system are constantly exposed to the aromas of the operational environment. In some embodiments, the exposure of the plurality of sensors to the aroma may be intermittent, after an interval, wherein the interval may depend on the processing and sensor cleaning capacity of the sensing system.

The plurality of sensors of the sensing system may be made from a polymeric material, wherein the polymeric material may comprise conducting particles. The polymers may be configured in the form of polymeric thin films, wherein the thin films may be deposited by processes such as drop casting, direct write, etc.

In some embodiments of the invention, each of the plurality of sensors of the sensing system may be made from a different polymeric material. In yet some further embodiments, some of the sensors of the sensing system may be formed from the same polymeric material, but with different concentrations of fillers. The polymers of the sensors may be made from at least one block polymer, such as polymers belonging to one polystyrene, poly siloxane, poly acetate, saccharides, and poly ether families. In an embodiment of the invention, the polymer of at least one of the sensors of the sensing system may be made from one of Polystyrene-co-methyl styrene, Poly (dimethyl siloxane-co-diphenyl siloxane), Poly (vinyl acetate), Polystyrene-co-Acrylonitrile, Hydroxypropyl cellulose or Poly (methyl vinyl ether-co maleic acid).

The conductive particles embedded in the polymer of the sensor may be one of carbon black and carbon nanotubes, wherein the weight ratio of conductive particles to polymer in the sensor may be 10% to 30%.

The sensing system may comprise an apparatus that includes a plurality of sensors that may be mounted on a single substrate. Further, the sensing system may comprise a plurality of electrodes, environmental sensors, a heater module, a clock or timer, a processor, a power supply, pumps and a communication module.

One end of the electrodes of the sensing system may be embedded in the polymer of the sensor of the sensing system. Another end may be connected with the power supply and processor of the sensing system, directly or indirectly. The electrodes of the sensing system may be used to transfer electrical energy from the power source to the sensor. The power may be transferred continuously or intermittently. Further, the electrodes may be used to communicate at least one of physical or chemical change, that the polymer of the sensor and thus the sensor underwent, to the processor when the sensor is exposed to the aroma of the operational environment.

In some embodiments, the sensing system may further comprise environmental sensors, such as temperature sensors, pressure sensors, humidity sensors, wind speed sensors etc. The data from at least one of the sensors of the environmental sensors may be provided to the processor of the sensing system.

In some embodiments, the clock or timer of the sensing system is used to record the time at which at least one of physical or chemical change is observed in the sensor element. Further, the clock or timer may be used to record the time of recording data from the plurality of environmental sensors of the sensing system. The processor of the sensing system may combine the time data with the sensing data of a plurality of sensors of the sensing system and the environmental sensors of the sensing system, thus making the data time-stamped data. In an embodiment of the invention, a separate processor may be provided that combines the data of the plurality of sensors of the sensing system and the environmental sensors, before sending it to the processor of the sensing system.

In some embodiments of the invention, a heater may be provided for each of the sensors of the sensing system, wherein the heater may heat the substrate of the sensing system and evaporate the aroma from the polymer of the sensors, thereby cleaning the sensor.

The sensing system may be provided with a power supply that provides electrical power to the plurality of sensors of the sensing system. The power supply may be an AC power supply, that may provide sinusoidal power to the plurality of sensors. In an embodiment, a DC power source is used to power the plurality of sensors of the sensor array, thereby providing constant power to the plurality of sensors of the sensing system.

The processor of the sensing system may be an artificial intelligence-based processor that comprises a plurality of trained models to infer the composition of the aroma from the time-stamped data from the plurality of sensors of the sensing system and the environmental sensors.

The communication module of the sensing system may be a wireless communication module, that comprises a transmitter and receiver working on WiFi, Bluetooth, 2G, 3F, LTE, 5G or other similar protocols. The wireless module may be used to communicate with a remote server, wherein the remote server may be provided with an artificial intelligence-based processor trained to infer the composition of the aroma from the time-stamped data from the plurality of sensors of the sensing systems and the environmental sensors. In some embodiments, a wireline network comprising fibre or electrically-conducting cables may be used to connect the sensing system with one of the nodes of one of the access networks, aggregate network or core network, to connect the sensing system with the remote server.

In some embodiments of the invention, the polymer of the plurality of sensors of the sensing system may undergo at least one of physical change or chemical change when it is exposed to components of the aroma. In an embodiment of the invention, the polymeric thin films may expand, and the resistivity of the polymeric thin film may be changed due to exposure to a reactive component. The electrodes embedded in the polymer are used to detect the change in at least one of the physical or chemical properties across the polymer and communicate with the processor, to provide at least one of the changes in physical and chemical properties to the processor.

In an embodiment of the invention, the electrodes detect and communicate at least one of change in electrical resistance between the electrodes, change in capacitance between the electrodes, heat generated across the polymer, or luminescence of the polymer, to the processor.

The processor of the chemical sensing system infers the plurality of components of the aroma, from the response of the plurality of sensors of the sensor array, by employing a plurality of artificial intelligence models trained to infer a plurality of components of an aroma, wherein at least one model is trained to detect at least one component of the aroma and wherein the model is trained to detect the response of the sensors that is not associated with the component of the aroma. At least one of the artificial intelligence models may be a long-short term memory (LSTM) network-based model, that learns the temporal sensor response for inference generation.

The processor infers the plurality of components of the aroma by passing the sensor response, generated during exposure of aroma to the sensor, through the artificial intelligence models that compare the response that is associated with the component and response that is not associated with the component and generate a confident inference about the presence of a component in the aroma.

From the response of the plurality of sensors of the sensing system and the plurality of environmental sensors, which may be time-stamped, a multi-dimensional response may be created by the processor, in the numeric space. Each model of the processor may use a plurality of sub-models to process the temporal data from the plurality of sensors of the sensing system and create a response corresponding to the components of the exposed aromas.

At least one of the sub-models of the plurality of sub-models from the models of the processor may be used to generate a feature representation of the temporal data from the plurality of sensors of the sensing system and the environmental sensors, wherein a change in a location of a point in the feature representation corresponds to the concentration of a component in the aroma.

In some embodiments, at least one of the sub-models of the plurality of sub-models from the processor may be used to generate a latent representation of the feature representation, thereby reducing the dimensionality of the temporal response of the sensing system.

In some embodiments, at least one of the sub-models of the plurality of models from the processor may be used to generate a straightened orthogonal representation from the generated latent representation, and convert the straightened orthogonal representation to the output representation, that corresponds to the concentration of components in the aroma. The straightened orthogonal representation may be transferred to a long-short term memory network-based model for inference generation.

In some embodiments, each output from the plurality of models may represent the presence of a functional group in the aroma, thus representing the component of the aroma. The output from at least one of the models may be represented in a plurality of different dimensions, to obtain a plurality of characteristic responses from the models, wherein each of the plurality of characteristic responses in a different dimension may represent the presence of the functional group in the aroma.

In some embodiments, the output from the combination for the plurality of models may be represented in a plurality of different dimensions, before generating a straightened orthogonal representation and corresponding output representation, to identify the presence and concentration of the functional group in the aroma and thus the composition of the aroma. In some embodiments, one (latent representation) in a first different dimension may provide the characteristic representation of ketone and one (latent representation) in a second different dimension may provide the characteristic representation of benzene. In some embodiments, the output from the combination of a plurality of models may be represented in a combination of a plurality of different dimensions, to obtain a characteristic representation of a functional group present in the aroma.

Each model of the processor may be statistically trained on test data to generate a representation, wherein the statistical training may be performed using a mixture of components to form an aroma or a single component.

In some embodiments, the output from the combination of at least one of the plurality of models may be scaled before feeding the representation to another model or representing the output in a plurality of other dimensions, to obtain a characteristic representation that may correspond to a functional group.

The deduced presence of a functional group from the processor may then be used by the processor to identify the component of the aromas, as each of the plurality of components of the aroma may have a characteristic functional group.

In some embodiments, the output representation of models corresponding to the concentration of functional group in an aroma may represent the concentration of a functional group present in the aroma and a concentration of a functional group not present in the aroma, wherein the plurality of models of the processor may be trained on a sample, wherein labels from the sample contain information about what is not present in the aroma, thereby increasing the confidence level of component deducing by the chemical sensing system.

In some embodiments, the output of some representations of the models may be used to deduce the presence of at least one of mould, virus, bacteria or fungus in the operational environment. The processor uses the output from the plurality of models, at different stages of processing, to relate the output with the presence of at least one of mould, virus, bacteria or fungus in the operational environment. In some embodiments, the output representation from at least one of the models may be stored in a memory and compared with a map by the processor to deduce the presence of at least one of mould, virus, bacteria or fungus in the operational environment.

In some embodiments, the sensing system may be provided with response or representation maps of at least one of mould, virus, bacteria and fungus or a combination thereof in the operational environment, wherein the dimensionality of at least one of the maps may be equal to the dimensionality of at least one of output representation of the plurality of models in the processor.

The processor may then compare the output representation of the plurality of models and the plurality of maps to deduce the presence of at least one of mould, virus, bacteria or fungus in the operational environment.

In some embodiments of the invention, a particulate monitoring sensor may be provided along with the sensing system, and the time-stamped data form the particulate monitoring sensor may be provided to the processor, along with the time-stamped data from a plurality of sensors of the sensing system and the environment sensors for deducing the presence of at least one of mould, virus, bacteria or fungus in the operational environment of the sensing system.

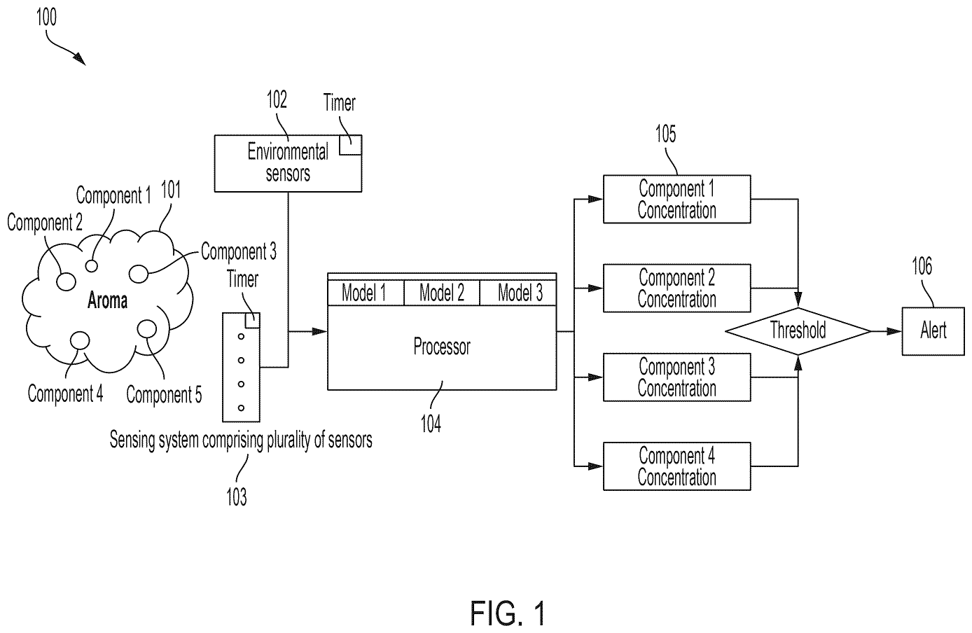

FIG. 1 depicts a process flowchart 100 of the chemical sensing system, according to one of the embodiments of the invention. The plurality of sensors of the sensor array present on a chip 103, are exposed to aroma (e.g., plume) 101, that includes a plurality of components or gases. The sensor array present on the chip may further include a timer. The response from the sensors (e.g., chemically-sensitive sensors) is merged with the environmental sensors 102 response and synchronized through a timer present on both of the units. The merged response is provided to a processor 104, that executes processor executable instructions to process the merged response using a plurality of models, examples of which are described herein. In some embodiments, the models generate a response regarding the presence and absence of a component from the aroma. In another embodiment, the models generate a response about the concentration of the component present in the aroma. In yet another embodiment, the system may be provided with an alert system 106, that issues a notification when a component of the aroma is above a predetermined threshold.

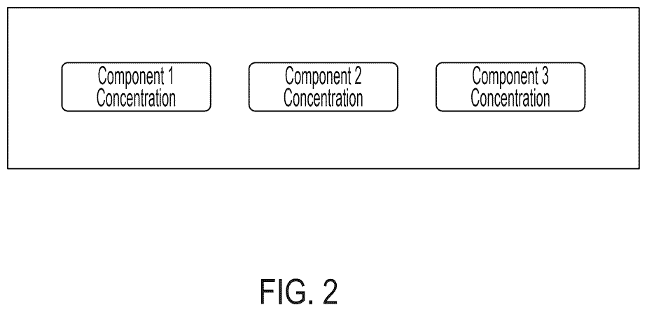

FIG. 2 depicts example aroma components information, generated by the chemical sensing system as per an embodiment of the chemical sensing system, in the form of a display. The information generated by the sensing system is depicted in the form of each component that the system can detect. Further, the presence and absence of a component is identified through the concentration of the component, as detected by the sensing system.

The environmental sensors of the sensing system may be used to provide sensing conditions to the processor, which allows the plurality of models and hence the processor to nullify the effects of environmental conditions on the output for the processor of the sensing system, thereby essentially rejecting the effect of environmental background on the sensitivity and functionality of the sensing system.

In some embodiments, the output of the processor may be averaged and presented to a user using a user interface. The averaging may be executed over an interval, to obtain a regularised response from the processor.

The user interface may be a graphical user interface, wherein the interface is provided through programs loaded in a user's smartphone, laptop, desktop, notebook, control centre, etc. The user interface may be used to view the concentration of components in the aroma of the operational environment. Further, the user interface may suggest the possible reason for the presence of a component in the aroma of the operational environment and/or a possible mitigation strategy to prevent an increase in the concentration of a component in the aroma of the operational environment. In some embodiments, the aroma may be air and the components may be one of COX, NOx, NH₃ etc.

In some embodiments of the invention, the sensing system may generate an alert if the concentration of a component of the aroma is deduced to be above a threshold level, wherein the processor may be provided with threshold level values, to compare with the deduced values.

In some embodiments, the alert may be provided in the form of a notification on the user's electronic device or an emergency alarm. In some embodiments, the alert may trigger an action-based control for the operational environment of the sensing system, wherein the action-based control may correspond to at least one of control of a heating ventilation and air conditioning (HVAC) unit, a purifier, a home appliance (e.g., a refrigerator), or an air recirculation unit.

Further, the sensing system may be employed in a home, with an air purifier, HVAC units, hospitals, factories, manholes, farms and animal husbandries. The action-based control may trigger the warnings and control the plurality of functional units of the operational environment of the system to mitigate the rise of a component in the aroma.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel. The process flowchart of a chemical sensing system depicted in FIG. 1 is similarly intended to be exemplary.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Even though particular combinations of features are recited in the claims and/or disclosed in the specification, these combinations are not intended to limit the disclosure of the specification. In fact, many of these features may be combined in ways not specifically recited in the claims and/or disclosed in the specification. Although each dependent claim listed below may directly depend on only one other claim, the disclosure of the specification includes each dependent claim in combination with every other claim in the claim set.

No element, act, or instruction used in the present application should be construed as critical or essential unless explicitly described as such. Also, as used herein, the article “a” is intended to include one or more items. Where only one item is intended, the term “one” or similar language is used. Further, the phrase “based on” is intended to mean “based, at least in part, on” unless explicitly stated otherwise.

1. A single-chip sensing system for detecting a plurality of components of an aroma, the system comprising:
a sensor array including a plurality of sensors, wherein at least one sensor of the plurality of sensors is configured to react with at least one of the plurality of components of the aroma and generate a response; and
a processor programmed to execute a plurality of artificial intelligence (AI) models trained to infer the plurality of components of the aroma from the response of the plurality of sensors of the sensor array, wherein at least one model of the plurality of AI models is trained to detect at least one component of the aroma, and wherein the at least one model is trained to detect the response of the sensors that are not associated with the component of the aroma,
wherein the plurality of AI models are trained to compare the response associated with a component and response that is not associated with the component and generate a confident inference about the presence of the component in the aroma.

2. The single chip sensing system of claim 1, wherein at least one model of the plurality of AI models comprises at least three sub-models that sequentially process at least a portion of the response from the plurality of sensors, to detect at least one component of the plurality of components of the aroma.

3. The single chip sensing system of claim 2, wherein a first sub-model of the at least three sub-models is trained to generate a feature representation of at least a portion of the sensor response, a second sub-model of the at least three sub-models is trained to generate a latent representation of the feature representation of the at least a portion of the sensor response, and a third sub-model of the at least three sub-models is trained to create a straightened orthogonal representation of the latent representation.

4. The single chip sensing system of claim 3 wherein at least one model of the plurality of AI models is trained to detect at least one of the plurality of components of the aroma from the straightened orthogonal representation by using a long short-term memory network-based model.

5. The single chip sensing system of claim 1 wherein the plurality of sensors of the sensor array are chemo-resistive sensors, each of the chemo-resistive sensors comprising a composite including a polymeric composition and conducting nanoparticles, and wherein an amount of polymer in the composite and an amount conducting nanoparticles in the composite is provided to at least one of the plurality of AI models, to assess the response of the sensors, using the plurality of AI models, wherein at least one of the plurality of AI models is trained to associate a response of the sensor with a component of the aroma, through the composition of the composite of the sensor.

6. A single chip sensing system configured to detect a plurality of components of a gaseous sample, the system comprising:
a sensor array comprising a plurality of sensors, wherein at least one sensor of the plurality of sensors is configured to react with at least one of the plurality of components of the gaseous sample and generate a response, and wherein the plurality of sensors of the sensor array are configured to react with each of the components of the plurality of components of the gaseous sample; and
a processor programmed to execute a plurality of models trained to detect the plurality of components of the gaseous sample from the response of the plurality of sensors of the sensor array,
wherein a temporal sensor response created by a change in a collective response of the plurality of sensors of the sensor array, is provided to the processor, and
wherein each of the models of the plurality of models is trained to associate a portion of the temporal sensor response with a component of the gaseous sample, thereby detecting the plurality of components of the gaseous sample.

7. The single chip sensing system of claim 6, wherein at least one model of the plurality of models of the processor comprises at least three sub-models, that sequentially process at least a portion of the response from the plurality of sensors, to detect at least one component of the plurality of components of the gaseous sample.

8. The single chip sensing system of claim 7, wherein a first sub-model of the at least three sub-models is trained to generate a feature representation of at least a portion of the sensor response, a second sub-model of the at least three sub-models is trained to generate a latent representation of the feature representation of the at least a portion of the sensor response, and a third sub-model of the at least three sub-models is trained to create a straightened orthogonal representation of the latent representation.

9. The single chip sensing system of claim 8, wherein at least one model is trained to detect at least one of the plurality of components of the gaseous sample from the straightened orthogonal representation by using a long short-term memory network-based model.

10. The single chip sensing system of claim 6, wherein the plurality of sensors of the sensor array are chemo-resistive sensors, each of the chemo-resistive sensors comprising a composite including a polymeric composition and conducting nanoparticles, and wherein an amount of polymer in the composite and an amount conducting nanoparticles in the composite is provided to at least one of the plurality of models, to assess the response of the sensors, using the plurality of models, wherein at least one of the plurality of models is trained to associate a response of the sensor with a component of the gaseous sample, through the composition of the composite of the sensor.
